(19) United States
(12) Patent Application Publication
(10) Pub. No.: US 2024/0148996 A1
(43) Pub. Date: May 9, 2024

(54) ARRANGEMENT IN THE MANNER OF A SPEECH VALVE FOR PLACING AND FITTING ON A TRACHEOSTOMY CANNULA

(71) Applicant: Albert-Ludwigs-Universit¿t Freiburg, Freiburg (DE)

(72) Inventors: Claudius STAHL, Heuweiler (DE); Klaus-Michael LÜCKING, Freiburg (DE); Falk TAUBER, Freiburg (DE); Thomas SPECK, Schallstadt (DE); Auth PHILIPP, Freiburg (DE); Noah KNORR, Freiburg (DE)

(21) Appl. No.: 18/550,513

(22) PCT Filed: Mar. 23, 2022
(86) PCT No.: PCT/EP2022/057574

§ 371 Date: Sep. 14, 2023

(30) Foreign Application Priority Data
Mar. 24, 2021 (EP) 21164568.4

(51) Int. Cl. A61M 16/04; A61M 16/20

(52) U.S. Cl. CPC A61M 16/0468; A61M 16/201; A61M 16/209; A61M 2205/0216; A61M 2205/3341; A61M 2205/3592; A61M 2210/1032

(57) ABSTRACT

A speech valve for placing and fitting on a tracheostomy cannula, having a throughflow body enclosing a throughflow channel which on one side at one end has a connection for joining to a proximal end of the tracheostomy cannula, a one-way valve located at an end of the throughflow body opposite the connection, and, at a distance along the throughflow channel between the connection and the one-way valve, a valve opening is located which is closable by an overpressure valve. The overpressure valve arrangement has at least one elastically deformable valve flap which, in a first state covers the valve opening provided between the connection structure and the one-way valve on the throughflow body and, at a predefinable overpressure occurring inside the throughflow channel converts, by an overpressure-driven elastic deformation of the valve flap, to a second state, in which it at least partially exposes the valve opening.

### CROSS-REFERENCE TO RELATED APPLICATIONS

Reference is made to PCT/EP2022/057574 filed Mar. 23, 2022, and European Patent Application No. 21164568.4 filed Mar. 24, 2021, which are incorporated herein by reference in their entirety.

### BACKGROUND OF THE INVENTION

### Field of the Invention

The invention relates to an arrangement in the manner of a speech valve for placing and fitting on a tracheostomy cannula, with a throughflow body enclosing a throughflow channel, which at one end has a suitably designed connection structure for joining to a proximal end of the tracheostomy cannula, at the end of which, opposite the connection structure along the throughflow channel, a one-way valve is arranged, and which provides between the connection structure and the one-way valve a valve opening that can be closed off by an overpressure valve arrangement.

### Description of the Prior Art

Tracheostomy cannulas serve to provide direct access to a person's trachea, bypassing the mouth, pharynx, and larynx, for the purpose of a direct air supply to, and removal from, the lungs. In addition to so-called unblocked tracheostomy cannulas, which are mainly used in spontaneously breathing patients with constant, reflexive and aspiration-free swallowing, so-called blocked tracheostomy cannulas, which provide a dilatable cuff on the distal side, are used for at least one of invasive ventilation, and ventilation of patients at risk of aspiration. It must be ensured that uncontrolled escape of respiratory gases through the pharynx and oral cavity is prevented during ventilation and, in addition, it must be prevented that, in particular in patients with dysphagia, food residues, or saliva, pass unhindered into the lower airways or into the lungs.

The open proximal end of a tracheostomy cannula has a suitably designed connection flange for the attachment to, for example, a ventilator, a cannula extension, or for the placement of a throughflow body that heats and humidifies the inhaled air. In short, it is an HME (heat and moisture exchanger), or a so-called speech valve.

A speech valve enables speech, provided that the vocal cords and the upper airway are intact and functional. A speech valve is a one-way valve that is designed to open when the patient breathes in, directing airflow to the lungs via the tracheostomy cannula, and to close when the patient breathes out, directing exhaled air through the larynx via the vocal cords, stimulating the latter to generate sound. However, this requires that the tracheostomy cannula is unblocked, that is to say, the cuff located at the distal end of the tracheostomy cannula must be emptied, so that a sufficiently large intermediate gap can form between the cuff and the trachea.

Alternatively or in combination with the unblocked tracheostomy cannula measure, so-called screened or fenestrated tracheostomy cannulas support a flow of exhaled air over the vocal cords during exhalation when using a speech valve.

However, a problem arises when using a speech valve with a non-fenestrated tracheostomy cannula if the cuff is inadvertently not emptied, that is to say, if the tracheostomy cannula is in a blocked state, so that there is no pathway for the patient's exhaled air. In this case, the speech valve allows inhalation but not exhalation. Within a few breaths, the lungs are over-inflated to a maximum extent. On the one hand, the patient is at risk of suffocating because he or she can no longer breathe, and on the other hand, there is an acute danger of the lungs bursting (pneumothorax), for example as a result of excessive overpressure when coughing.

A similar situation can occasionally occur with an unblocked tracheostomy cannula, if the mucose membrane around the tracheostomy cannula swells unexpectedly, thereby narrowing the lumen between the trachea and the tracheostomy cannula.

To counteract these dangers, to which a patient in particular is exposed when a speech valve is directly placed on a blocked, and neither fenestrated nor screened, tracheostomy cannula, it is proposed in WO 2016/139441 A1 that an overpressure valve is arranged in the region of the speech valve attachment between the connection to the tracheostomy cannula and the one-way valve of the speech valve attachment; the function of the valve is set such that the overpressure valve closes in a manner impermeable to fluid or gas within the speech valve arrangement at pressure conditions that are not dangerous for the patient, and is only able to trigger spontaneously in the event that a predefined overpressure is exceeded within the speech valve arrangement, as a result of which a valve opening is freed, through which excess exhaled air can flow out into the environment largely without resistance.

The overpressure valve of known art is designed in the form of a ball valve, which, by virtue of the self-weight of the ball, is able to close a valve opening in a manner impermeable to gas. Here the size, weight and arrangement of the ball are dimensioned such that in the event of an overpressure situation that is dangerous for the patient, the ball is lifted against the force of gravity, as a result of which the vent opening is freed.

The speech valve for tracheostomy cannulas described in EP 2 908 895 B1 also has, in addition to the one-way valve function characteristic of speech valves, the possibility of venting by manual transference of an opening mechanism into an open position in which free breathing is enabled, as is the case with a tracheostomy cannula without a speech valve fitted. Opening requires an active, purposeful action on the part of the patient, which cannot necessarily be assumed to be possible in the case of patients receiving intensive care.

A combined valve arrangement for attachment to a tracheostomy cannula is described in the document EP 2 326 376 B1; this features an inspiration valve and a separate expiration valve for the purpose of artificial, that is to say. mechanical ventilation, wherein the latter is designed as an adjustable PEEP (positive end-expiratory pressure) valve. In particular, the expiration PEEP valve takes the form of a ball and cage valve with a spring, the tension of which can be used to adjust the airflow resistance of the expiration PEEP valve.

The document EP 0 617 630 B1 discloses a tracheostoma valve that features a combined inspiration and expiration valve, both of which are in the form of planar closing elements that can be pivoted bidirectionally about an axis on one side. The two closing elements are able to close the tracheostoma valve completely in a mutually overlapping manner, and to open, partially or completely, the air flow path, depending on the air pressure conditions inside the valve.

EP 0078685 A1 describes an arrangement with a valve for controlling the flow of air through the tracheostoma in response to respiratory airflow pressure, together with a device for securing the valve in a releasable manner to the stoma. The valve has a flexible diaphragm that can be moved between a first, a second and a third position, wherein the first position is used for normal breathing through the valve; the flexible diaphragm is moved by vocal exhalation into the second position, in which it engages with the valve seat, thereby interrupting the air flow through the valve, and the flexible diaphragm is moved into the third position, by pressure generated in the trachea as a result of coughing, so as to allow an air flow through the valve.

US Published patent application 2014/0305440 A1 discloses a ventilation valve that is connected to a tracheostomy cannula and has a tubular valve body, at the proximal end of which is detachably inserted a disc endpiece fitted with openings, and with an O-ring around its circumference. A slack diaphragm, which lies over the inner face of the disc endpiece, serves as an inlet valve to allow patient inhalation and air intake, and as a check valve to block patient exhalation, directing it to the patient's larynx, sinuses, and mouth to allow normal speech. The O-ring gives way if the patient exhales forcefully, so as to free the disc endpiece from the tubular valve body. The ventilation valve itself is also attached to the tracheostomy cannula in order to prevent it from slipping after disconnection. The inhalation valve also has a whistle that produces an audible signal if exhalation is forceful.

Despite the availability of speech valves on the market, together with the knowledge and informal advice that speech valve attachments should only be placed on unblocked tracheostomy cannulas, deaths still occur worldwide due to the placement of a speech valve attachment on a blocked tracheostomy cannula, even in cases when experienced surgeons or care workers are fitting the cannula. As a consequence, in July 2019 the Federal Institute for Drugs and Medical Devices (Bundesinstitut für Arzneimittel and Medizinprodukte) referred to this defect in fitness for purpose, and mandated clear warning labels on speech valve attachments. Even the use of overpressure valves designed as ball valves in the region of the speech valve arrangement cannot rule out functional failures, due to their sensitivity to spatial position.

### SUMMARY OF THE INVENTION

The invention is a speech valve for placement and fitting onto a tracheostomy cannula, with a throughflow body that encloses a throughflow channel, and at one end has a suitably designed connection structure for joining to a proximal end of the tracheostomy cannula, at the end of which, opposite the connection structure along the throughflow channel, a one-way valve is arranged, and, between the connection structure and the one-way valve, an opening is provided that can be closed off by an overpressure valve arrangement, such that the risk to the patient that has existed up to now when a speech valve is placed on or against the tracheostomy cannula can be eliminated, or significantly reduced. Thus, on the one hand, the aforementioned speech valve should ensure that an overpressure that spontaneously builds up in the airway of a patient, for example as a result of coughing, is safely and immediately relieved, and this in every conceivable spatial position of the speech valve arrangement, that is to say, independent of the direction of the force of gravity. As an alternative to, or in combination with, the above provision, it is necessary to modify or supplement the speech valve arrangement such that, in the event of a life-threatening overpressure situation that leads at least to a partial opening of an existing overpressure valve arrangement, the care worker who is concerned with the speech valve arrangement is alerted to what could otherwise be a life-threatening situation for the patient.

In accordance with the invention, an arrangement in the manner of a speech valve for placement and fitting onto a tracheostomy cannula, is designed such that the overpressure valve arrangement has at least one elastically deformable valve flap which, in a first state—in which pressure conditions prevail within the speech valve such as are usual when speaking, and, in the event of a predefinable overpressure within the flow channel, can be converted by an overpressure-driven, elastic deformation of the valve flap into a second state that at least partially exposes the valve opening—in which overpressure conditions prevail, such as occur, for example, when coughing.

The design and the functional principle of the overpressure valve arrangement attached to the throughflow body of the speech valve is inspired by an opening mechanism found in nature on a suction trap used by the carnivorous plant “water hose” (Utricularia vulgaris/Utricularia australis). The suction trap takes the form of a bladder trap that can be closed with a flexible flap and is able to transport liquid in the form of water from the inside of the bladder trap via glands into the environment, whereby the walls of the bladder trap bulge inwards, forming an elastic wall tension, wherein the entrance to the trap is closed in a watertight manner by a trap door, which in this state is bulged outwards and rests with its free edge against a woven step. In this state, there is an underpressure inside the trap compared to the ambient pressure. Upon contact with a prey, the trap door reverses its convex curvature into a concave shape within less than 2 ms, which frees the trap entrance, at least in some regions. In this unclosed suction trap state, an underpressure-driven pressure equalization takes place, whereby the suction trap walls relax and water and prey flow into the interior of the suction trap directly in front of the suction trap opening, by virtue of the increase in volume that occurs within a millisecond.

The overpressure valve arrangement integrated in the arrangement within a speech valve makes use of the functionality explained above in an abstracted manner (reversed with respect to the pressure conditions) in that the at least one elastically deformable valve flap, corresponding, as it were, to the trap door in the biological model, assumes the second state, at least partially releasing the valve opening, as soon as a sudden pressure difference occurs between the interior of the throughflow body, that is to say, the speech valve arrangement, and the ambient pressure, which usually corresponds to the currently prevailing air pressure, the at least one elastically deformable valve flap assumes the second state, which at least partially exposes the valve opening, whereby the overpressure is instantaneously reduced.

In order not to impair the functionality of the speech valve in the speech range, the overpressure-dependent opening of the overpressure valve arrangement, which is implemented by an almost instantaneous transfer of the at least one elastically deformable valve flap from the first to the second state, is to be initiated by an adjustable pressure difference, for example, from 35 to 45 cm H₂O, that is to say, from 0.034 bar to 0.044 bar. In the pressure range below the above trigger pressure for the overpressure valve arrangement, that is to say, for pressures in the speech range between 0 and 25 cm H₂O, that is to say, between 0 and 0.0245 bar, the at least one elastically deformable valve flap remains in the first, that is to say, closed, state, so that a sufficiently pressurized air flow is able to stimulate the patient's own vocal cords to speak during normal operation of the speech valve. The actual pressure ranges to be applied and the maximum opening area of the valve flap can be implemented differently depending on the muscle strength, size, and lung volume of the patient, preferably by choosing a lower overpressure range and a smaller maximum opening area for weak or small patients with a low lung volume, than for strong or large patients with a high lung volume.

In contrast to a ball valve arrangement of known art, the functioning of which depends on the self-weight of the ball and, associated with this, on the spatial arrangement and position of the entire ball valve, the self-weight of the at least one elastically deformable valve flap plays no role, or no significant role, in the functioning of the overpressure valve arrangement in accordance with the invention, as a result the functioning of the arrangement in accordance with the invention within the speech valve remains ensured in any spatial arrangement and position.

The throughflow body has a throughflow channel wall radially encompassing the throughflow channel which is locally penetrated by the valve opening of the overpressure valve arrangement. The valve opening is spaced along the throughflow channel, preferably centrally between the connection structure and the one-way valve. The valve opening can be assigned an opening normal, which is oriented orthogonally to the longitudinal axis of the throughflow channel, that is to say, the valve opening is oriented towards the longitudinal axis of the channel.

In a preferred form of embodiment, a single elastically deformable, planar valve flap is provided, which covers the valve opening in the first state, wherein the elastically deformable valve flap is joined directly or indirectly to the throughflow body relative to the valve opening, such that the elastically deformable valve flap is arranged facing towards the throughflow channel and away from the valve opening. In an alternative form of embodiment, the valve flap is arranged externally to the throughflow body, and in the first state closes the valve opening on the rear face. This ensures that in the event of damage, the valve flap, or even parts thereof, cannot enter the patient's airway. In addition, the valve flap can be replaced quickly and safely in the form of a replacement module, without at the same time having to open the interior of the speech valve.

In all possible examples of embodiments, two half-spaces can be assigned to the valve opening, of which in one of the two half-spaces the elastically deformable valve flap is joined, at least in some sections, in a fixed position relative to the valve opening, and within the other of the two half-spaces the elastically deformable valve flap is mounted in a slack manner relative to the valve opening. In a figurative sense, the joining of the elastically deformable valve flap relative to the valve opening corresponds to an attachment of a swing door such that it can be rotated on one side of a door frame. In contrast to the attachment and design of a swing door, known per se, the at least one elastically deformable valve flap in the first state covers the valve opening within its second half-space, at least in some sections, with an edgewise excess length, that is to say, the elastically deformable valve flap extends, at least in some sections, beyond a region of the valve opening edge. This edgewise excess length contributes to the elastically deformable valve flap remaining within the first state, that is to say, in the state closing the valve opening, for as long as the pressure prevailing within the speech valve arrangement remains below the predefined overpressure range.

In addition to the dimensioning of the excess length, many other material and design parameters play an important role in the adjustment of the overpressure-driven opening function of the innovatively designed overpressure valve arrangement. As will be explained further with reference to illustrated examples of embodiments, the design, size, and choice of material for the design of the at least one elastically deformable valve flap, as well as the geometry of the edgewise valve opening contour, contribute to a safe and reliable functionality of the overpressure valve arrangement.

The edgewise excess length is preferably not of the same dimension, but has a minimum of at least one point. In this way, the opening procedure can be determined by the overpressure-driven elastic deformation of the at least one valve flap in a geometrically predefined manner, in which the at least one valve flap implements an elastic transformation from the first to the second state as rapidly as possible.

By virtue of the typically cylindrical design of the throughflow body of known per se speech valve attachments on tracheostomy cannulas, it makes sense to design the valve opening, spaced, preferably centrally, between the connection structure and the one-way valve, as a rectangular or slot-shaped recess within the throughflow body wall, or as a valve opening that adjoins the rectangular or slot-shaped recess within the throughflow body wall, and whose larger opening dimension is arranged in the circumferential direction around the throughflow channel, and whose smaller opening dimension is arranged parallel to the throughflow channel.

Here the at least one elastically deformable valve flap is of planar design, and has a planar curved shape that is appropriately adapted so as to cover the valve opening.

In order to take into account the aforementioned aspect of different speech pressures in patients of different constitution, the at least one elastically deformable valve flap can be replaceably attached directly or indirectly to the throughflow body of the arrangement, in the manner of a speech valve. In one variant of embodiment, the elastically deformable valve flap is detachably attached on one side by two attachment pins, to which the valve flap can be attached to form a planar, curved, spatial shape. In this case, the two attachment pins face towards the throughflow channel in the region of the overpressure valve arrangement. In an alternative example of embodiment, the overpressure valve arrangement connects externally to the throughflow body in the region of the valve opening, and provides a slot-shaped insertion opening, into which the valve flap can be inserted in the form of a plug-in module. In this form of embodiment, the valve flap can, for reasons of hygiene, be switched externally, without having to open the speech valve. For this purpose, a holder for the detachable securement of the valve flap is removed; the latter can then be pulled out of the slot-shaped insertion opening, and replaced by a new valve flap. Any accidental slippage and inhalation of the valve flap is prevented by the holder, together with the standard particle filter foam element in the speech valve.

By keeping in stock a number of valve flaps that differ from each other, at least in terms of their shape, size or elastic properties, a patient-specific selection can be made in order to be able to select the trigger pressure of the overpressure valve arrangement in a patient-specific manner. Further details can be found later in the description with reference to an illustrated example of embodiment.

As an alternative to, or in combination with, the design of the aforementioned safe overpressure valve arrangement that opens in a manner that is independent of position, the risk to the patient's life resulting from an incorrect operation of a speech valve arrangement can be avoided, or significantly reduced, by arranging a signal generator indirectly or directly on the overpressure valve arrangement, or downstream of it, however the overpressure valve arrangement may be designed. This generates a signal in the event of an overpressure-driven opening of the overpressure valve arrangement, that is to say, in the overpressure range.

The signal generator is preferably designed to generate an acoustically perceptible sound signal, in the form of an alarm signal that can be unmistakably perceived by a person who is managing the speech valve arrangement. The alarm signal alerts the person to a possible fault, so that the fault of a blocked tracheostomy cannula can be corrected immediately.

At the same time as the signal is generated, the pressure is spontaneously relieved by the open overpressure valve arrangement, preventing the risk of immediate asphyxiation and bursting of the patient's lungs. The person managing the speech valve attachment is thus given time to correct the operating fault. The combination of signal generation with the function of pressure relief, preferably on the basis of the above-described overpressure valve arrangement, designed in accordance with the solution, ensures on the one hand the undisturbed function of the speech valve in normal use, that is to say, with normal breathing and speaking pressures prevailing (speaking range), without false alarms, and on the other hand, if a predefined overpressure range is exceeded within the speech valve attachment, both a triggering of a signal and an immediate relief of pressure are implemented at the same time for the protection of the patient. If, after triggering, that is to say, opening of the overpressure valve arrangement, the pressure prevailing in the throughflow body is reduced, that is to say, for example, by inhalation, the elastic valve flap swings back into its initial position and closes the valve opening automatically, so that the speech valve can continue to be used in the usual way.

In a preferred form of embodiment, the signal generator is designed in the form of a whistle, and is located within a throughflow channel downstream of the overpressure valve arrangement. Depending on the design and arrangement of the whistle, the throughflow channel can be structured locally so as to form a labial or lingual whistle.

Alternatively, or in combination, the signal generator is able to generate an electrical, electromagnetic, or optical, signal, which is either emitted in the immediate vicinity for physiological perception by a person, or is transmitted by cable or wirelessly to a monitoring unit, for example in the form of a central monitoring unit. For this purpose, a sensor system detecting the overpressure situation is to be arranged in or on the overpressure valve arrangement; its signals can be fed to a central monitoring unit so as to trigger an alarm, or can be transformed into a physiologically perceptible form. The sensor system can be combined with the valve mechanism, or implemented as an independent signal transmitter.

### BRIEF DESCRIPTION OF THE DRAWINGS

Without any limitation to the general concept of the invention, the invention is described below by way of examples of embodiment with reference to the figures.

FIG. 1 shows a longitudinal-sectional view in perspective of a first arrangement designed in accordance with the invention, in the manner of a speech valve;

FIGS. 2A-F show cross-sectional views of the first arrangement of an overpressure valve arrangement in accordance with the invention, as a sequence of diagrams;

FIGS. 3A-J show longitudinal-sectional views in perspective through a first arrangement designed in accordance with the invention, in the manner of a speech valve, as a sequence of diagrams;

FIG. 4 shows a cross-sectional perspective view of the first arrangement designed in accordance with the invention, in the manner of a speech valve;

FIG. 5 shows a longitudinal-sectional perspective view of a second arrangement designed in accordance with the invention, in the manner of a speech valve;

FIG. 6 shows a longitudinal-sectional perspective view rotated through 90° relative to FIG. 5, of the second arrangement designed in accordance with the invention, in the manner of a speech valve;

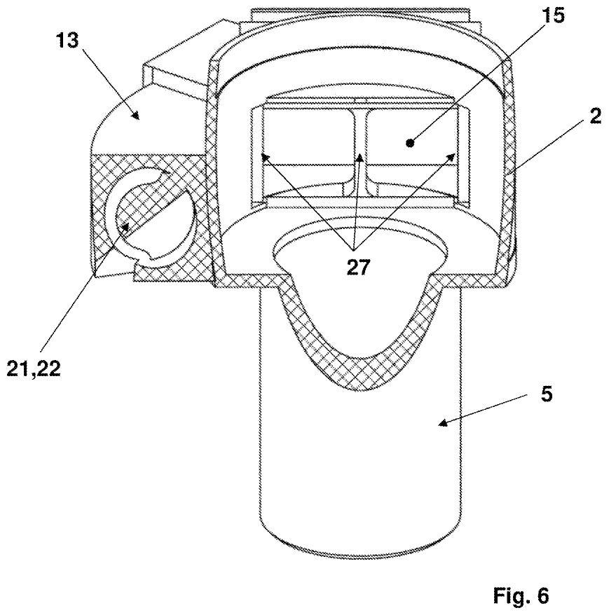

FIG. 7 shows an oblique perspective view of the second arrangement designed in accordance with the invention, in the manner of a speech valve, without a one-way valve; and

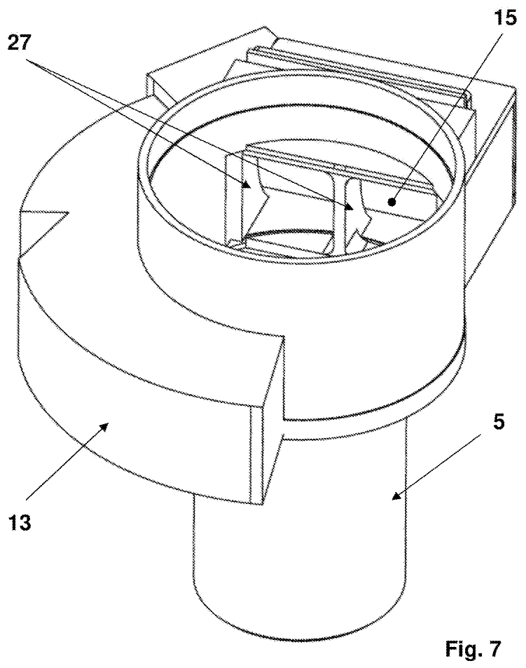

FIGS. 8A-F show a perspective longitudinal-sectional view through the overpressure valve arrangement of the second arrangement designed in accordance with the invention, in the manner of a speech valve, as a sequence of diagrams.

### DETAILED DESCRIPTION OF THE INVENTION

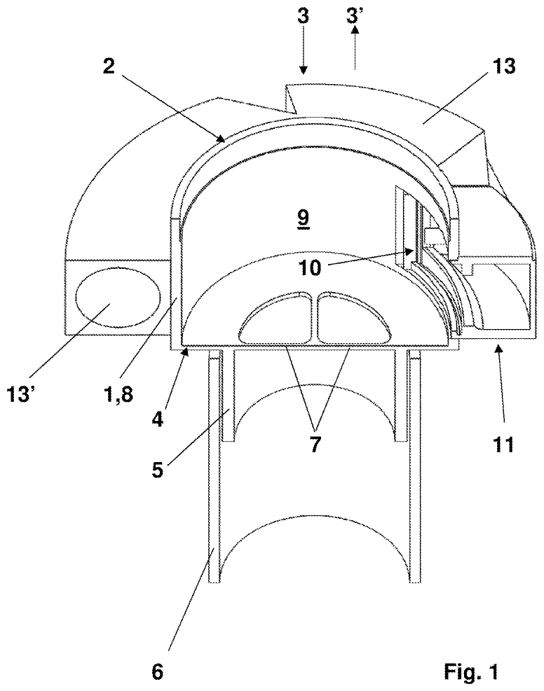

FIG. 1 shows a longitudinal-sectional perspective view of a speech valve arrangement that has a hollow, cylindrical throughflow body 1, at the upper throughflow body end 2 of which, open in FIG. 1 in the interests of a better presentation, a one-way diaphragm flap valve, known per se, is arranged, which opens the upper throughflow body end for an inspiratory air inflow 3 and prevents a respiratory air outflow 3′.

The lower end of the throughflow body 4 in FIG. 1 is connected to a connection structure 5, via which the speech valve arrangement can be joined to a tracheostomy cannula 6, preferably in a removable and fluid-impermeable manner. At the transition between the throughflow body 1 and the connection structure 5, which has a smaller internal diameter than the throughflow body 1 in the example of embodiment shown, a screen structure 7 is arranged which, on the one hand, has a protective function against uncontrolled entry of foreign particles in the direction of the tracheostomy cannula 6 and, on the other hand, provides a mechanically stable support plane for a standard particle filter foam element (not shown).

In addition, the arrangement in the manner of a speech valve provides within the flow body 1, wall 8 which surrounds a throughflow channel 9, an opening 10, which completely penetrates the throughflow body wall 8, and is arranged between the upper end of the throughflow body 2 and the lower end of the throughflow body 4.

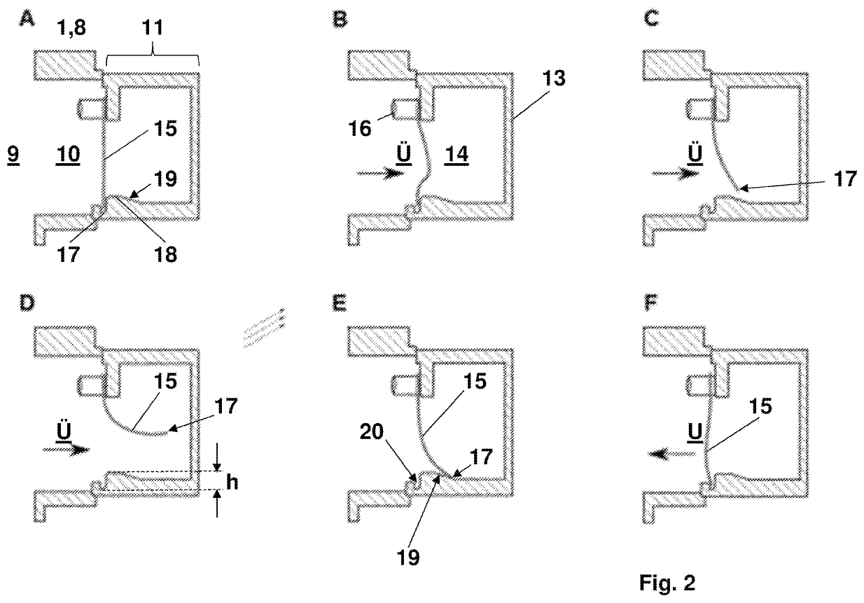

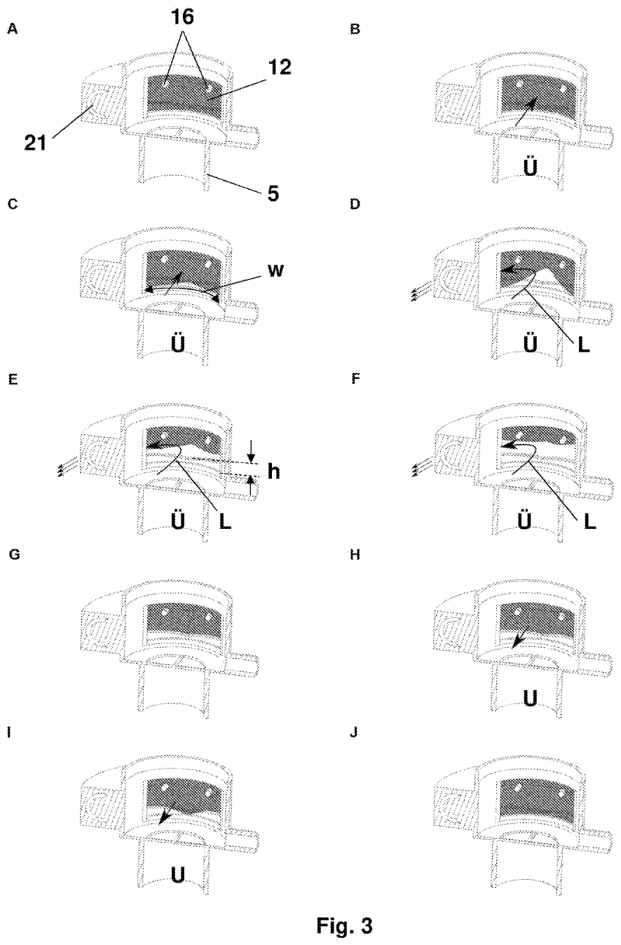

An overpressure valve arrangement 11 is fitted to the opening 10 having a construction and operation which is explained in more detail below with reference to the sequence of diagrams that can be found in FIGS. 2 and 3.

The sequence of diagrams in FIGS. 2A to F in each case represent longitudinal sections through the overpressure valve arrangement 11 arranged on the opening 10 within the wall 8 of the throughflow body 1. The throughflow body wall 8 radially surrounds the throughflow channel 9. The sequence of diagrams shown in FIGS. 3A to J represent perspective longitudinal sections through the entire arrangement in the manner of a speech valve. All the sequences of diagrams illustrated in FIGS. 2 and 3 are able to characterise in a comprehensible manner the mode of operation and the dynamic deformation behavior of the elastically deformable valve flap 15 that determine the overpressure valve function.

The overpressure valve arrangement 11, directed radially external to the throughflow body 1, connects to the opening 10 inside the throughflow body wall 8 in a fluid-impermeable manner. The overpressure valve arrangement 11 can be a separate unit from the throughflow body 1, as shown, or can be integrally connected to the latter. The overpressure valve arrangement 11 has a valve opening 15, which, in the case shown, is of smaller dimensions than the opening 10 projecting through the throughflow body wall 8. However, variants of embodiment for the overpressure valve arrangement 11, in which the opening 10 and the valve openings 12 are identical, are also possible.

The overpressure valve arrangement 11 has a throughflow channel wall 13 surrounding a throughflow channel 14, which opens into the environment via at least one opening 13′, see FIG. 1. In this manner, ambient pressure conditions prevail within the throughflow channel 14.

In a first state, see FIGS. 2A, 3A, the valve opening is completely covered by an elastically deformable valve flap 15. The valve flap 15 is of a membrane- or film-like design and consists, for example, of an elastomer. Two attachment pins 16 are used to attach the valve flap 15 within the overpressure valve arrangement 11; these are attached to the overpressure valve arrangement 11 in a radial projection onto the opening 10 within the opening region of the opening 10, see in particular the representation in FIGS. 3A to J. The elastically deformable valve flap 15 has holes corresponding to the attachment pins 16, so that the valve flap 15 is fixedly joined to the overpressure valve arrangement 11 in a removable manner along its upper valve flap edge.

The shape and size of the elastically deformable valve flap 15 are adapted to the shape and size of the valve opening 12 such that it projects axially on both sides of the valve opening 12 with an excess length, whereas the extent of the valve flap 15 in the circumferential direction corresponds at most to the valve opening width w of the valve opening 12, see FIG. 3C. The joining of the elastically deformable valve flap 15 to the overpressure valve arrangement 11 also enables an uncomplicated replacement of the valve flap 15.

In a non-joined state, the design of the elastically deformable valve flap 15 has a planar rectangular shape. After joining the valve flap 15 into the overpressure valve arrangement 11 by means of the attachment pins 16, the valve flap 15 is transformed into a planar curved shape that is appropriately adapted to the cylindrical curvature of the flow body wall 8, whereby the elastically deformable valve flap 15 obtains a dimensional stability co-determined by the curvature, its size and shape, and the inherent elasticity of the valve flap material.

The lower valve flap edge 17 of the valve flap 15, which is located axially opposite to the attachment pins 16, projects axially beyond the lower edge bounding the valve opening 12, which has a step-like design, see FIGS. 2A to F in this regard. The step-like design of the lower valve opening edge 18 provides an axially oriented flank facing towards the valve flap 15 in the first state, against which the lower valve flap edge 17 lies flush with an excess length. In the direction of the inner throughflow channel 14 of the overpressure valve arrangement 11, there is an adjoining, continuously falling, step flank 19, the function of which will be explained later. In addition, the step-shaped lower valve opening edge 18 has a varying step height h in the circumferential direction of the overpressure valve arrangement 11, such that the step height h is a maximum at both valve opening edges located opposite each other in the circumferential direction, and is a minimum at the center, see FIGS. 2D, 3E,F. In this way, the elastically deformable valve flap 15 overlaps the valve opening 12 in each case with a maximum excess length in its edge regions, and with a minimum excess length in the central region of the valve flap 15.

The shape, size, thickness, and elastomeric material of the elastically deformable valve flap 15 determine the elastic deformation behavior of the valve flap, and thus determine the minimum overpressure required to open the overpressure valve assembly. By the provision of a number of variously designed valve flaps, which differ from each other in their elastic deformation behavior, the arrangement in the manner of a speech valve, in accordance with the invention, can be individually adapted to the physiological ventilation capabilities and characteristics of a patient.

Furthermore, reference is made to the sequence of diagrams in both FIGS. 2 and 3 to explain in more detail the transition of the resiliently deformable valve flap 15 from the first state, in which the overpressure valve arrangement 11 is closed, to the second state, in which the overpressure valve arrangement 11 assumes an open position.

FIGS. 2A, 3B show the overpressure valve arrangement 11 in the first state, that is to say, the state in which the valve flap 15 closes off the valve opening 12 in a largely gas-impermeable manner and ensures that pressure conditions can develop within the throughflow body 1 that enable the patient to speak.

If the pressure inside the throughflow body 1 increases, for example due to spontaneous coughing, the elastically deformable valve flap 15 is deformed in an overpressure-driven manner, see FIGS. 2B, 3B. At a sufficiently high overpressure U acting on the valve flap 15 from the inside, that is to say, from the side of the throughflow channel 9, the valve flap 15 is deformed radially outwards by the pressure event, primarily in the central region, so that the lower freely movable valve flap edge 17 brushes over the lower step-like valve opening edge 18, which is lowered centrally relative to the edge regions. In this opening moment of the overpressure valve arrangement 11, a central vertical fold is formed on the lower valve flap edge 17 by virtue of the step-like geometry along the lower valve opening edge 18, see FIG. 3C, whereby the valve flap 15 deforms radially outwards from its center in a sudden or abrupt manner into the volume of the overpressure valve arrangement 11, see FIGS. 3D, 2C. At this moment, there is both a temporary stiffening of the valve flap 15 at its center and a structural instability of the surface of the valve flap 15 due to the removal of its surface curvature over the entire valve flap 15. Furthermore, the valve flap 15 is displaced, driven by overpressure, from its center into the lumen, that is to say, into the throughflow channel 14 of the overpressure valve arrangement 11, whereby the edgewise regions of the valve flap are pulled back from the lower valve opening edge 18. The valve opening 12 is freed as shown in FIGS. 2C, 2D, and 3E, F respectively, whereby the overpressure U formed abruptly within the throughflow body 1 is effectively relieved by an air flow L directed through the valve opening 12.

When the overpressure is depleted, that is to say, when the pressure is fully equalized, the elastically deformable valve flap returns to its original position by virtue of its inherent elasticity, see FIGS. 2E, 3G, and its lower valve flap edge 17 comes into contact with the obliquely inclined step flank 19 of the lower valve opening edge 18, see FIG. 2E. If there is no further pressure surge triggered by coughing, the valve flap 15 is returned to the first state, in which the valve flap 15 completely covers the valve opening 12, if necessary, supported by an underpressure U forming inside the throughflow body 1 as a result of inhalation, as shown in FIGS. 2F, 3J.

In a preferred embodiment, as shown in the sequence of diagrams in FIGS. 2A to F, the lower valve flap edge 17 opens into a groove-shaped recess 20, whereby in addition to the inherent stability of the valve flap 15 in the first state by virtue of its curvature, the possibility of movement of the valve flap 15 is limited. Both the curved shape of the valve flap 15, which is caused by the cylindrical or round shape of the throughflow body, and a particle filter foam element (not shown) sitting on the screen structure 7, as well as the joining of the valve cap 15 within the groove-shaped recess 20, prevent the valve flap 15 from being pushed through, or sucked into, the interior of the speech valve arrangement.

The valve flap principle, which has been borrowed from the field of biology, as explained earlier, is based exclusively on an isotropically acting pressure difference between the interior of the throughflow body 1 and the environment. In this manner, the mode of operation of the “bionically borrowed” overpressure valve arrangement 11 is independent of position.

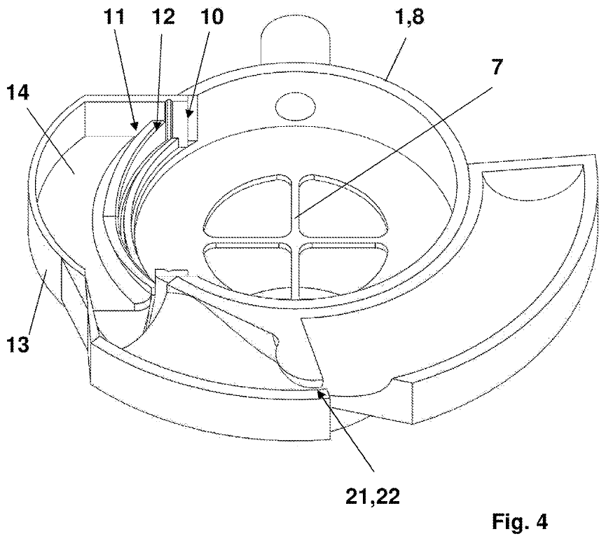

In a preferred development of the arrangement of a speech valve, to counter the risk of a life-threatening situation for the patient as a result of improper handling of the tracheostomy cannula, a signal generator 21 for the generation of an acoustically perceptible sound signal, preferably in the form of a whistle 22, is arranged downstream of the overpressure valve arrangement 11, as shown in FIG. 3 and FIG. 4. FIG. 4 shows a cross-section through the throughflow body 1, together with the overpressure valve arrangement 11. Downstream of the opening 10, that is to say. the valve opening 12, is a throughflow channel 14, which, in the case of the embodiment shown in FIG. 4, is oriented in the circumferential direction of the throughflow body 1. A whistle 21, preferably in the form of a labial whistle contour, is arranged along the throughflow channel 12. If an overpressure situation endangering the patient occurs, the overpressure valve arrangement 11 is opened abruptly, for example in the aforementioned manner, whereby an escape of the overpressure-driven air flow through the overpressure valve arrangement 11 in the region of the whistle 22 generates an acoustically perceptible signal tone, which alarms the surrounding environment, so that a person caring for the patient is immediately made aware of the dangerous situation and can take appropriate countermeasures.

Not necessarily, but advantageously, the signal generator 21 is combined with the aforementioned bionic overpressure valve in the manner shown in FIG. 4. Similarly, overpressure valve arrangements known per se, for example in the form of a ball valve, can be provided with an appropriate signal generator, preferably in the form of a whistle, in the region of the air outlet opening.

It is also possible to design the signal generator to generate an electrical, electromagnetic, or optical, signal, which can be transmitted to a central monitoring unit, where appropriate by use of a cable, or wirelessly. Sensor systems known per se to the person skilled in the art are available for this purpose, with which, for example, at least one of a deflection of the elastically deformable valve cap, and an air flow within the throughflow channel, can be detected.

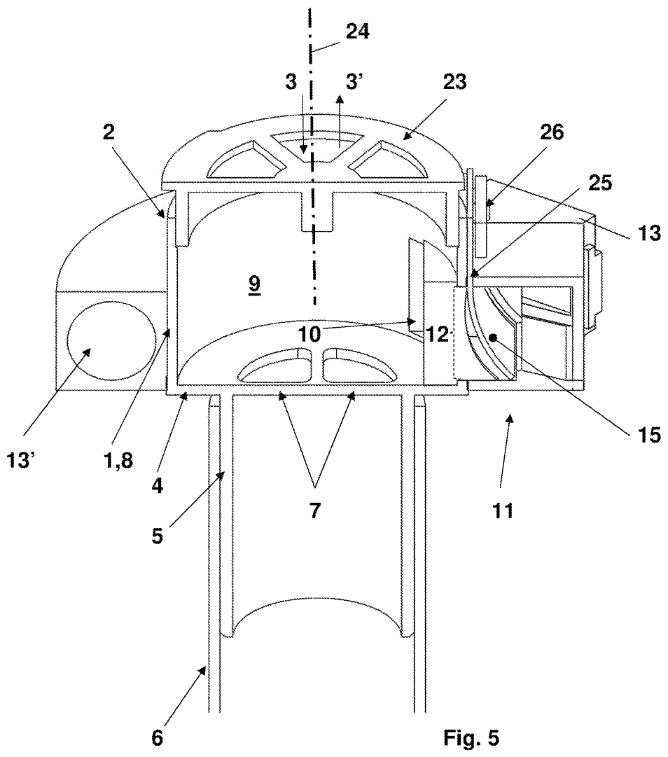

FIG. 5 shows a second example of embodiment of a speech valve arrangement in accordance with the invention, which is shown in a perspective longitudinal-sectional view in the same manner as that in FIG. 1. In order to avoid repetition, the aforementioned reference symbols that are also used in what follows, correspond to the components as described above, the functions of which are not explained again. In an addition to FIG. 1, FIG. 5 shows that a one-way diaphragm flap valve of known art is arranged at the upper, open end of the throughflow body 2, which opens the upper end of the throughflow body 2 for an inspiratory air inflow 3, and closes it for a respiratory air outflow 3′.

In addition, in the arrangement as a speech valve within the throughflow body 1, the throughflow body wall 8 of comprises a throughflow channel 9, providing an opening 10 that completely penetrates the throughflow body wall 8, and is arranged between the upper throughflow body end 2 and the lower throughflow body end 4, that is, the opening 10 is arranged along the throughflow channel 9 at a distance from the upper and lower throughflow body ends 2, 4 respectively, and is oriented towards the throughflow channel 9 transversely to the longitudinal extent 24 of the latter.

An overpressure valve arrangement 11 is connected to the opening 10 externally to the throughflow body wall 8. Its construction and mode of operation is basically similar to that of the overpressure valve arrangement 11 illustrated in FIGS. 1 to 4. In contrast to the latter, however, the valve flap 15 (FIG. 5) does not face towards the throughflow channel 9, and is detachably attached by two attachment pins 16, as can be seen in FIGS. 2A to F, and 3A to J. Instead the valve flap 15 can be inserted as a plug-in module into a slot-shaped recess 25 inside the throughflow channel wall 13 of the overpressure valve arrangement 11, and can be fixed to the outside of the throughflow body wall 8 by use of a holder 26. In this way, the valve flap 15 in the first state is able to cover or close the valve opening 12 of the overpressure valve arrangement 11, which is directly radially outwardly adjacent to the opening 10 projecting through the throughflow body wall 8, from the side facing away from the throughflow channel, in which pressure conditions are formed inside the throughflow body 1 that enable the patient to speak. The attachment of the valve flap 15 externally to the throughflow body 1 prevents the valve flap 15 from detaching in an uncontrolled manner and entering the throughflow channel 9.

In addition, the overpressure valve arrangement 11 has curved baffle walls 27 in the center and at the edge of the valve opening 12, against which the valve flap 15 clings in the first state, and in this way assumes a defined valve flap curvature, which, as in the biological model of the trap door system of the suction trap of the water hose (genus Utricularia)—as explained earlier is convexly curved towards the side of the acting pressure, that is to say, towards the speech valve body. The radius of this curvature is determined by the baffle walls.

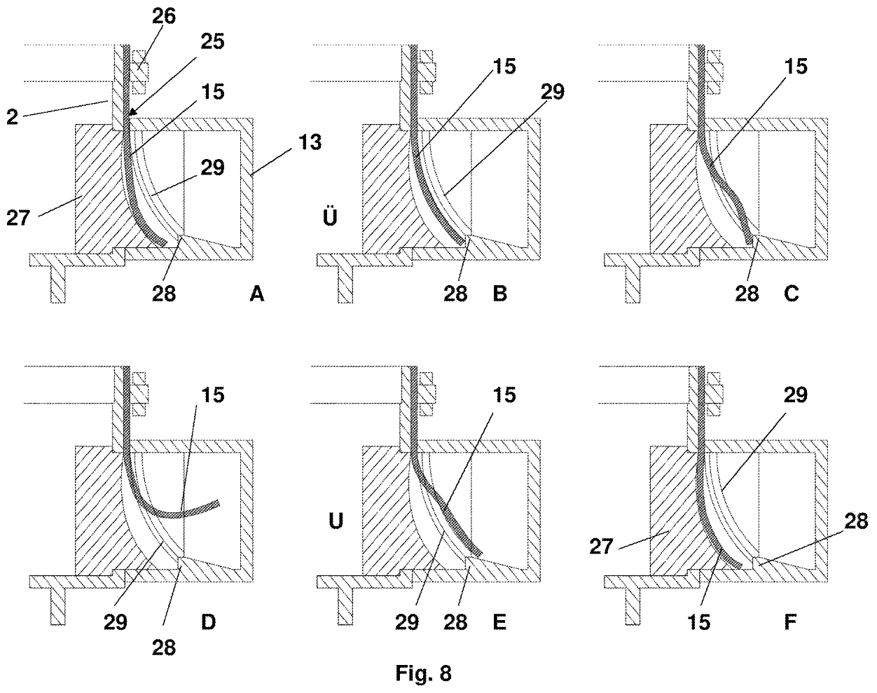

Reference is further made to the sequence of diagrams A to F in FIG. 8 in order to explain in more detail the transfer of the resiliently deformable valve flap 15 from the first state, in which the overpressure valve arrangement 11 is closed, to the second state, in which the overpressure valve arrangement 11 assumes an open position, and back.

In the quiescent state, see FIG. 8A, the valve flap 15 sits on the baffle walls 27. A step structure 28 is also provided at the lower edge of the valve opening 12. Lateral step structures 29 are provided on each of the two side walls laterally bounding the valve opening 12. In the case of normal speech, the valve flap 15 is deflected from its quiescent position. See FIG. 8B, which the lower edge of the valve flap 15 sits against the inside of the lower step structure 28, as in the biological model. In order to prevent leakage in this closed valve position, both side edges of the valve flap 15 also fit tightly against the side step structures 29 from the inside.

In the event of an abrupt pressure event within the speech valve body, for example one that is initiated by coughing, there is a change in the curvature of the valve flap 15, preferably in the center of the planar valve flap 15 as shown in FIG. 8C, whereupon the valve flap 15 swings past the step structures 28 and 29 and the valve opening 12 subsequently opens completely, see FIG. 8D.

During a subsequent inspiration by the patient, an underpressure (U) is created in the interior of the speech valve, which causes the valve flap 15 to seal against the rear of the step structures 28, 29 as shown in FIG. 8E, in order subsequently to return to its initial position, see FIG. 8F. In order to ensure the inspiration-driven return, the step structures 28, 29 are in each case flattened at the side of the step facing away from the valve flap in the initial state.

Downstream of the overpressure valve arrangement 11, as in the speech valve arrangement in the example of embodiment illustrated in FIGS. 1 to 4, a signal generator 21 is arranged for the generation of an acoustically perceptible sound signal, preferably in the form of a whistle 22, so that reference is made to the above description for a commentary on this subject. In the case of the above overpressure situation, in which the overpressure valve arrangement 11 opens completely, see FIG. 8D, the pressure compensation-driven air flow passing through the throughflow channel 14 generates an unmistakable signal tone as it passes the whistle 2.

The dynamic deflection behavior of the valve flap, both in the event of the valve opening and in the event of the return of the opened valve flap into the closed position, can be influenced by an initial curvature of the valve flap as well as by the material and shape properties of the planar, elastic valve flap, such as thickness, stiffness, and elasticity of the valve flap, which is preferably made of an elastomer material. If stiffer or softer, or thicker or thinner, valve flaps are used, the valve opens at a lower or a higher pressure. Depending on the type of patient (man/woman/child) and/or the physiological constitution of the patient, a suitable valve flap must be inserted into the slot-like insertion opening of the overpressure valve arrangement provided for this purpose. For this purpose, the attending physician can make use of a number of variously designed valve flaps, which are designed and stocked as replaceable insert modules.

### LIST OF REFERENCE SYMBOLS

1 Throughflow body
2 Throughflow body end
3 Inspiratory air inflow
3′ Respiratory air outflow
4 Lower throughflow body end
5 Connection structure
6 Tracheostomy cannula
7 Screen structure
8 Throughflow body wall
9 Throughflow channel
10 Opening
11 Overpressure valve arrangement
12 Valve opening
13 Throughflow channel wall
13′ Opening of the throughflow channel
14 Throughflow channel
15 Valve flap
16 Attachment pin
17 Valve flap edge
18 Lower valve opening edge
19 Step flank
20 Groove-shaped recess
21 Signal generator
22 Whistle
23 One-way flap valve arrangement
24 Longitudinal extent of the throughflow channel
25 Slot-shaped recess
26 Attachment element
27 Baffle walls
28,29 Step structures
L Air flow
Ü Overpressure
U Underpressure, inhalation

1-18. (canceled)

19. A speech valve for use in a tracheostomy tube comprising:
a throughflow body enclosing a throughflow channel, one end of the throughflow channel including a connection joining the one end of the throughflow body to a proximal end of the tracheostomy tube, a pressure responsive one way valve including at least one opening located in the throughflow channel, the pressure responsive one way valve including at least one deformable valve flap which in a first pressure state covers the at least one opening and in a second pressure state is elastically deformed to at least partially expose the at least one opening to permit air flow through the speech valve.

20. A speech valve in accordance with claim 19, comprising:
a signal generator located on the overpressure responsive one way valve, or is located downstream of the overpressure responsive one way valve, wherein the signal generator generates a signal in response to an overpressure causing opening of the overpressure valve.

21. A speech valve in accordance with claim 20, wherein:
the signal generator generates an acoustically perceptible sound.

22. A speech valve in accordance with claim 21, wherein:
the signal generator generates the sound signal by vibrational excitation of an air flow passing through the overpressure valve.

23. A speech valve in accordance with claim 21, comprising:
the signal generator being located downstream of the valve opening and being turned off by the overpressure valve.

24. A speech valve in accordance with claim 21, wherein:
the signal generator is a whistle.

25. A speech valve in accordance with claim 24, wherein:
the whistle is a labial or lingual whistle.

26. A speech valve in accordance with claim 20, wherein:
the signal generator generates an electrical, electromagnetic, or optical signal and the signal is transmitted to a monitor.

27. A speech valve in accordance with claim 25, wherein:
the throughflow channel is bounded by a throughflow channel wall or by a throughflow body and a throughflow channel wall.

28. A speech valve in accordance with claim 19, wherein:
the at least one elastically deformable valve flap is at least one of being shaped or being elastic for developing a restoring force by the elastically deformable valve flap being at least partially transferred from the second state into the first state.

29. A speech valve in accordance with claim 19, comprising:
two half-spaces located in the valve opening; and
within one of the two half-spaces, at least in some sections of the half-spaces, the elastically deformable valve flap is fixed to the valve opening and the elastically deformable valve flap is mounted in an other of the two half-spaces to be slack relative to the valve opening.

30. A speech valve in accordance with claim 19, wherein:
the at least one elastically deformable valve flap is at least one of replaceably joined to the throughflow body and the overpressure valve or external to the throughflow body.

31. A speech valve in accordance with claim 19, wherein:
at least two elastically deformable valve flaps are stored prior use and selected to differ from each other at least in one of shape, size, and elastic properties and at least one of the two valve flaps are joinable together by at least one of the throughflow body and to the overpressure valve as a function of physiological ventilation characteristics of a patient.

32. A speech valve in accordance with claim 29, wherein:
within the second half-space, the at least one elastically deformable valve flap in the first state covers the valve opening and at least in some sections thereof length of the edge housing causes the valve flap to extend, at least in some sections, beyond a valve opening width.

33. A speech valve in accordance with claim 32, wherein:
the edge housing has a minimum at at least one point of contact.

34. A speech valve in accordance with claim 19, wherein:
the at least one elastically deformable valve flap is planar and, in a first state, has a surface normal, subtending at least an angle α with a throughflow body a longitudinal axis associated with the throughflow channel, and this angle is defined as 80°≤α≤100°.

35. A speech valve in accordance with claim 19, wherein:
the throughflow body includes a throughflow body wall radially enclosing the throughflow channel and an opening of the one way valve completely penetrating the throughflow body wall.

36. A speech valve in accordance with claim 19, wherein:
the planar valve flap in the first state has a convex surface curvature orienting towards the throughflow channel.
